(19) United States
(12) Patent Application Publication
(10) Pub. No.: US 2023/0270169 A1
(43) Pub. Date: Aug. 31, 2023

(54) VAPORISER DEVICE, IN PARTICULAR A STERILISATION VAPORISER DEVICE, FOR VAPORIZING A LIQUID AND/OR AN AEROSOL

(71) Applicant: Ampack GmbH, Konigsbrunn (DE)

(72) Inventors: Korbinian Tomschi, Zahling (DE); Jürgen Haak, Sonnenbuehl (DE); Markus Zimmermann, Landsberg am Lech (DE)

(21) Appl. No.: 18/043,696

(22) PCT Filed: Sep. 6, 2021
(86) PCT No.: PCT/EP2021/074510

§ 371 Date: Mar. 1, 2023

(30) Foreign Application Priority Data
Sep. 8, 2020 (DE) 10 2020 123 389.2

(51) Int. Cl. A24F 40/46; A24F 40/10; A24F 40/53

(52) U.S. Cl. CPC A24F 40/46; A24F 40/10; A24F 40/53

(57) ABSTRACT

A vaporizer device for vaporizing a liquid and/or an aerosol, has at least one housing comprising at least one vaporizer space housing, which delimits a vaporizer space into which the liquid to be vaporized and/or the aerosol to be vaporized can be fed, and has at least one heating unit for a vaporization of the fed-in liquid and/or the fed-in aerosol, wherein the heating unit is realized as a matrix heating unit comprising a plurality of heating elements, which can be regulated and/or monitored separately, wherein the heating unit comprises the plurality of heating elements which are arranged irregularly and/or regularly in a plane, wherein the plurality of heating elements are arranged at a heating plate of the heating unit, wherein a surface of the heating plate forms a vaporizer surface of the heating unit for a vaporization of the liquid and/or of an aerosol.

### CROSS REFERENCE TO RELATED APPLICATIONS

This application is based on and incorporates herein by reference the German patent application 10 2020 123 389.2, filed on Sep. 8, 2020, and the international patent application PCT/EP2021/074510, filed on Sep. 6, 2021.

### BACKGROUND

From DE 10 2017 215 200 A1 a vaporizer device, in particular a sterilization vaporizer device, for vaporizing a liquid and/or an aerosol is already known, wherein the vaporizer device has at least one housing comprising at least one vaporizer space housing, which delimits a vaporizer space into which the liquid that is to be vaporized and/or the aerosol that is to be vaporized can be fed, and has at least one heating unit for a vaporization of the fed-in liquid and/or the fed-in aerosol.

Furthermore, from DE 10 2016 105 676 A1, DE 35 40 161 A1 and DE 10 2011 056 440 A1 vaporizer devices are already known.

### SUMMARY

The invention is based on a vaporizer device, in particular a sterilization vaporizer device, for vaporizing a liquid and/or an aerosol, in particular of liquid H2O2 and/or aerosolized H2O2, with at least one housing comprising at least one vaporizer space housing, which delimits a vaporizer space into which the liquid to be vaporized and/or the aerosol to be vaporized can be fed, and with at least one heating unit for a vaporization of the fed-in liquid and/or the fed-in aerosol, in particular in the vaporizer space.

It is proposed that the heating unit is realized as a matrix heating unit comprising heating elements, in particular heating circuits, which can be regulated and/or monitored separately, in particular individually, wherein the heating unit comprises a plurality of heating elements which are arranged irregularly and/or regularly in a plane, wherein the heating elements are arranged at a heating plate of the heating unit, wherein a surface of the heating plate forms a vaporizer sur-face of the heating unit for a vaporization of the liquid and/or of an aerosol. Preferably the heating elements of the matrix heating unit are divided, in particular segmented, in groups. The matrix heating unit may, in particular alternatively or additionally, be implemented as a heating unit comprising a single meander-like heating element, which is in particular arranged in a plane and which comprises several heating zones that are individually regulatable, or may comprise a plurality of meander-like heating elements, which are in particular in a plane and which are preferably divided, in particular segmented, in groups. Preferentially the matrix heating unit comprises a plurality of segmented electrical heating circuits which are, in particular viewed in a plane, arranged concentrically or in a grid. The heating elements, in particular the heating resistors, can be regulated and/or monitored separately, in particular individually. That the heating elements, in particular the heating resistors, “can be regulated and/or monitored separately, in particular individually” shall in particular define that the heating elements, in particular the individual heating resistors, can be regulated to a temperature independently from one another, and/or that a temperature of each individual heating element can be registered separately at each heating element, independently from the other heating elements. Preferably a heating element, in particular each separate heating element, forms an individual heating circuit of the heating unit, in particular in a circuitry context. The heating elements are preferentially arranged at, in particular in, a heating plate of the heating unit. The heating plate is preferably made of a ceramic or metallic material. It is however also conceivable that the heating plate is made of a different material, deemed expedient by someone skilled in the art, which is suitable for use in a vaporizer process, in particular in an H2O2 vaporizer process. The heating elements are preferentially arranged at the heating plate so as to be distributed in a regular or in an irregular manner. Preferably the heating elements are arranged spaced apart from each other at the heating plate, in particular viewed in a plane. In particular, the heating elements are arranged at the heating plate such that they are separated by webs of the heating plate. Preferably the heating elements are inserted, in particular embedded, in the heating plate. The heating elements may be realized in a one-part implementation with the heating plate, like for example cast into the heating plate or the like, or may be inlaid, in particular fixed, in receptacles, like for example grooves of the heating plate. The term “in a one-part implementation” is in particular to mean connected by substance-to-substance bond, like for example by a welding process and/or gluing process etc., and especially advantageously molded-on, like by a production from a cast and/or by a production in a one-component or multi-component injection-molding procedure. Preferably the heating elements terminate at least substantially flush with a surface of the heating plate. The sur-face of the heating plate, in particular together with a surface of the heating elements, forms a vaporizer surface of the heating unit for a vaporization of a liquid and/or of an aerosol, in particular of liquid H2O2 and/or aerosolized H2O2. It is however also conceivable that the heating elements are arranged at, in particular in, the heating plate in a plane that is arranged offset from and parallel to a surface of the heating plate that forms the vaporizer surface, and that the heating elements, for example, contact the vaporizer surface in order to heat it. Other arrangements of the heating elements at, in particular in, the heating plate, deemed expedient by someone skilled in the art, are also conceivable. The vaporizer device is preferably intended to be used in a sterilization process of packaging elements in production machines in the field of asepsis and/or ultra-cleaning.

The housing of the vaporizer device is preferably realized in a multi-part implementation. The housing is in particular configured for accommodating, at least partially enclosing and/or sup-porting individual components of the vaporizer device. “Configured” is in particular to mean specifically implemented, specifically designed and/or specifically equipped. By an object being configured for a certain function is in particular to be understood that the object fulfills and/or executes said certain function in at least one application state and/or operation state. The housing comprises the vaporizer space housing which delimits the vaporizer space wherein, in particular in a mounted state of the housing, one side of the vaporizer space is delimited by the heating plate. The vaporizer space housing is preferably realized in a bell shape. In particular viewed in a plane extending at least substantially perpendicularly to a longitudinal axis of the housing, the vaporizer space housing may have a round, elliptic or polygonal cross section or may have a different cross section deemed expedient by someone skilled in the art. The longitudinal axis of the housing preferably runs at least substantially perpendicularly to the vaporizer surface of the heating plate. The heating plate is preferably arranged at the housing symmetrically, in particular rotationally symmetrically, to the longitudinal axis. The heating plate in particular has a longitudinal extent that runs at least substantially perpendicularly to the longitudinal axis. The vaporizer surface preferably extends at least substantially perpendicularly to the longitudinal axis. The term “substantially perpendicularly” is here in particular intended to define an orientation of a direction relative to a reference direction, wherein the direction and the reference direction, in particular viewed in a projection plane, include an angle of 90°, the angle having a maximal deviation that is in particular smaller than 8°, advantageously smaller than 5° and especially advantageously smaller than 2°. The vaporizer space housing preferably delimits the vaporizer space at least along a circumferential direction and in at least one direction that runs at least substantially parallel to the longitudinal axis, in particular on a side of the vaporizer space that faces away from the heating plate. The circumferential direction preferentially runs in the plane extending at least substantially perpendicularly to the longitudinal axis of the housing. The vaporizer space housing is preferably made of a metallic material. It is however also conceivable that the vaporizer space housing is made of a different material deemed expedient by someone skilled in the art and is suitable to be used in a vaporizer process, in particular in an H2O2 vaporizer process.

The vaporizer device preferentially comprises at least one sealing unit, comprising at least one sealing element which is arranged between the vaporizer space housing and the heating plate. The sealing element may have a round or a polygonal cross section or a further cross section that is deemed expedient by someone skilled in the art. Viewed along a direction running at least substantially parallel to the longitudinal axis, the sealing element may be arranged between the vaporizer space housing and the heating plate or, viewed along a direction running at least substantially perpendicularly to the longitudinal axis, the sealing element may be arranged between the vaporizer space housing and the heating plate. The sealing element preferably adjoins with at least one side the vaporizer space housing and with at least one further side the sealing element adjoins the heating plate. The vaporizer space housing and/or the heating plate may have at least one receiving recess for receiving the sealing element. However, it is also conceivable that the sealing unit comprises a plurality of sealing elements which are arranged between the vaporizer space housing and the heating plate, in particular viewed along a direction running at least substantially perpendicularly to the longitudinal axis and/or along a direction running at least substantially parallel to the longitudinal axis.

The housing further comprises a heating housing, which is configured at least for accommodating the heating unit. The heating housing is preferably made of a metallic material. It is however also conceivable that the heating housing is made of a different material that is deemed expedient by someone skilled in the art. The heating housing and the vaporizer space housing are preferentially — in particular releasably — connected with each other. Preferably the heating housing and the vaporizer space housing each comprise a connection flange for a connection of the heating housing and the vaporizer space housing. In a connected state of the heating housing and the vaporizer space housing, the heating plate is arranged between the heating housing and the vaporizer space housing, in particular in a region of a connection interface. Preferably the vaporizer space housing is realized so as to be removable from the heating housing, in particular for maintenance purposes of the heating unit. The connection interface preferably extends in a plane extending transversally, in particular at least substantially perpendicularly, to the longitudinal axis.

The vaporizer device preferentially comprises at least one insulation unit having at least one insulation element, which is arranged between the vaporizer space housing and the heating housing, in particular for an insulation of electronic components of the heating unit, which are arranged in the heating housing, against a huge heat load. The insulation element may have a round or a polygonal cross section or a further cross section that is deemed expedient by some-one skilled in the art. Viewed in the direction that runs at least substantially parallel to the longitudinal axis, the insulation element is preferentially arranged between the vaporizer space housing and the heating housing, in particular in the region of the connection interface. Preferentially the insulation element adjoins the vaporizer space housing with at least one side and adjoins the heating housing with at least one further side. The insulation unit preferably comprises at least one further insulation element, which is arranged on an outer side of the vaporizer space housing. The further insulation element encompasses the vaporizer space housing along the circumferential direction. Preferentially the further insulation element adjoins an outer surface of the vaporizer space housing. The further insulation element is configured for an insulation of the vaporizer space, in particular in order to counteract a formation of condensate on an inner wall of the vaporizer space housing and/or to counteract a convection to the environment/ambient air. It is conceivable that the insulation unit alternatively or additionally comprises further insulation elements for an insulation of individual components of the vaporizer device.

The vaporizer device preferably comprises at least one feeding unit comprising at least one feeding element, in particular a feeding nozzle or a feeding dripper, for a feeding of a liquid and/or an aerosol, in particular of a liquid H2O2 and/or aerosolized H2O2, into the vaporizer space. The feeding element is preferably arranged at the vaporizer space housing, in particular at a cover of the vaporizer space housing which is situated opposite the heating plate. However, it is also conceivable that the feeding element is arranged in a different position of the housing, deemed expedient by someone skilled in the art, so as to allow a feeding of a liquid and/or an aerosol, in particular of a liquid H2O2 and/or aerosolized H2O2. The feeding element is preferentially arranged at the vaporizer space housing in such a way that a central axis of the feeding element is oriented at least substantially parallel to, in particular coaxially with, the longitudinal axis of the housing. The cover of the vaporizer space housing is preferably arranged, in particular fixed, in a removable manner on a base body, in particular a cylindrical, preferably circle-cylindrical, base body of the vaporizer space housing. However, it is also conceivable that the cover is realized in a one-part implementation with the base body of the vaporizer space housing, in particular free of a releasable connection technique, and/or is arranged on the base body in a loss-proof manner.

The vaporizer space housing preferably comprises a cleaning access opening or a cleaning connection for allowing a cleaning process of the vaporizer space, in particular a CIP process (Cleaning-in-Place process). The cleaning access opening or the cleaning connection may be oriented transversally, in particular at least substantially perpendicularly, or at least substantially parallel to the longitudinal axis of the housing. The cleaning access opening or the cleaning connection may, alternatively or additionally, comprise an overflow opening of the vaporizer space housing for conveying away a non-vaporized portion of the liquid fed to the vaporizer space and/or of the aerosol fed to the vaporizer space. The vaporizer space housing preferentially comprises at least one vaporizer outlet or a vaporizer outflow connection for conveying the liquid vaporized in the vaporizer space and/or the aerosol vaporized in the vaporizer space, in particular an H2O2 vapor-gas mixture, to a packaging element directly or via a distributor, in particular via one or several conduit/s of a production machine, in particular of a food product filling and/or food product packaging machine comprising the vaporizer device. The vaporizer outlet or the vaporizer outflow connection may be oriented transversally, in particular at least substantially perpendicularly, or at least substantially parallel to the longitudinal axis of the housing.

An implementation according to the invention allows achieving a high degree of process reliability in a vaporizer process, in particular a sterilization process. Advantageously individual actuation of respective heating elements is enabled. It is advantageously possible to obtain an even heating picture over a substantially entire operating time of the vaporizer device. It is advantageously possible to detect a formation of drops on the vaporizer surface and preferably to counteract this early in a vaporizer process. Advantageously selective energy input is made possible. Advantageously low energy consumption is attainable as a drop formation on the vaporizer sur-face can be advantageously counteracted, in particular as individual subregions of the vaporizer surface can be operated individually at different temperatures.

It is further proposed that each heating element is assigned an, in particular individual, regulation electronics element of the heating unit and/or an, in particular individual, monitoring electronics element of the heating unit, each heating circuit in particular comprising an integrated regulation electronics element of the heating unit and/or an integrated monitoring electronics element of the heating unit. The regulation electronics element and/or the monitoring electronics element of the heating unit are/is preferably arranged at least to a large extent in the heating housing on a side of the heating plate that faces towards the vaporizer space housing. However, it is also conceivable that the regulation electronics element and/or the monitoring electronics element of the heating unit are/is arranged outside the heating housing, being for example part of a computing unit that is external and/or is embodied separately from the heating unit, and which is for example configured for a controlling or regulation of further components. In particular, the regulation electronics element and/or the monitoring electronics element of the heating unit are connected to the heating elements, in particular in a manner already known to someone skilled in the art, by means of connection lines of the heating unit, in particular electrical connection lines of the heating unit. The connection lines may be embodied as cables, as soldered copper lines realized in a one-part implementation with the heating plate, or as different connection lines which are deemed expedient by someone skilled in the art. The regulation electronics element and/or the monitoring electronics element of the heating unit are/is preferentially configured to regulate and/or to monitor the heating elements separately, in particular individually. In particular, each individual heating element is assigned an individual regulation electronics element and/or an individual monitoring electronics element for the purpose of regulating and/or monitoring a temperature of the respective heating element separately, in particular individually. It is conceivable that the individual regulation electronics elements and/or the individual monitoring electronics elements are implemented as individual hardware components, or that the individual regulation electronics elements and/or the individual monitoring electronics elements are implemented as software components which are selectively assigned to the individual heating elements. A temperature monitoring and/or temperature regulation of the individual heating elements may be brought about, for example, by a monitoring/regulation of a resistance value of the heating elements, in particular of the heating elements which are embodied as heating resistors, or may be carried out in a different manner that is deemed expedient by someone skilled in the art. Alternatively, it is also conceivable that each individual heating element is assigned its own controlling electronics element of the heating unit for an individual controlling of a temperature of the heating elements. The implementation according to the invention allows attaining a high degree of process reliability in a vaporizer process, in particular in a sterilization process. It is advantageously possible to reliably detect deviations from set values of temperatures of the heating elements within a cycle period of the vaporizer process, in particular the sterilization process. Advantageous regulation with a small reactive power is enabled.

Furthermore, it is proposed that the heating elements are arranged concentrically around a central axis of the heating unit, or that the heating elements are arranged in a grid-like fashion on a side of the heating unit that faces toward the vaporizer space housing. Preferably, in particular in a state of the heating unit being arranged at the housing, the central axis of the heating unit runs at least substantially parallel to, in particular coaxially with, the longitudinal axis of the housing. The heating elements are preferably arranged concentrically around the longitudinal axis of the housing or mirror-symmetrically with respect to at least one plane that comprises the longitudinal axis completely. The heating elements may be realized as punctiform or as linear heating resistors. In an implementation of the heating elements as punctiform heating resistors, preferably several heating elements together form a group that can be regulated and/or monitored separately, in particular independently from other groups of heating elements. Preferably, in an implementation of the heating elements as punctiform heating resistors, the heating elements are for example arranged similar to a grid of a screen and preferably form individually actuatable heating pixels. In an implementation of the heating elements as linear resistors, these may be implemented and/or arranged, for example, in a circular-arc shape, in a straight line/straight lines, in an l-shape, in an s-shape, in a zigzag shape, in a wave pattern, in a row-and-line array, or the like. The heating elements may preferably be arranged so as to be distributed regularly in a plane extending at least substantially perpendicularly to the central axis of the heating unit, or may have dimensions which increase or decrease outward, towards an edge of the heating plate, depending on a distance from a center of the heating plate. Other implementations and/or arrangements of the heating elements, deemed expedient by someone skilled in the art, are also conceivable. An implementation according to the invention allows attaining a high degree of process reliability in a vaporizer process, in particular in a sterilization process. It is advantageously possible to obtain a large heated vaporizer surface for a vaporization, wherein the individual heating elements, which have high energy requirements, can be regulated individually for counteracting drop formation on the vaporizer surface and for detecting contamination.

It is moreover proposed that the housing comprises at least one, in particular the aforementioned, heating housing for an accommodation of the heating unit, wherein the heating housing is — in particular directly — connected with the vaporizer space housing, in particular via the aforementioned connection interface, and the heating elements are arranged at least partly in the region of a connection interface of the housing between the heating housing and the vaporizer space housing. Preferably a plane, which extends at least substantially parallel to the connection interface and at least substantially perpendicularly to the longitudinal axis, intersects with the heating unit. The heating unit is preferably surrounded by the vaporizer space housing and the heating housing along the circumferential direction, in particular in a state when the vaporizer space housing and the heating housing are arranged adjoining each other. Preferably the heating plate is — in particular directly — adjacent to the vaporizer space housing, in particular with an edge region of the heating plate. The implementation according to the invention allows attaining a high degree of process reliability in a vaporizer process, in particular in a sterilization process. An advantageous heat input into the vaporizer space is enabled. Easy maintenance of the heat-ing element is possible, in particular due to simple removal of the vaporizer space housing from the heating housing.

It is further proposed that, in particular in a state when the vaporizer space housing and the heating housing are arranged adjoining each other, the heating unit is arranged at the vaporizer space housing in such a way that at least one heating element of the heating unit is, in particular directly, adjacent to the vaporizer space housing. Preferably individual heating elements of the heating unit are at least partly adjacent to the vaporizer space housing. Preferentially individual heating elements contact the vaporizer space housing directly, in particular at least with subregions of the individual heating elements. The implementation according to the invention allows attaining a high degree of process reliability in a vaporizer process, in particular in a sterilization process. Advantageously, heating of the vaporizer space housing can be realized for the purpose of counteracting a formation of condensate on an inner wall of the vaporizer space housing.

Beyond this it is proposed that the vaporizer device comprises at least one elastic preload element, in particular a spring element, which is configured to subject the heating unit to a preload force toward the vaporizer space housing. The preload element is preferably arranged in the heating housing. The preload element is preferentially supported with one side on the heating unit and with a further side the preload element is supported at the heating housing, in particular at an inner wall of the heating housing. The preload element may be embodied as a helical spring, as an elastomer, as a gas compression spring, as a leaf spring, as a hydraulic piston, or as another preload element which is deemed expedient by someone skilled in the art. The implementation according to the invention allows attaining a high degree of process reliability in a vaporizer process, in particular in a sterilization process. It is advantageously achievable that the heating unit securely adjoins the vaporizer space housing so as to enable a reliable heat input into the vaporizer space housing.

Furthermore, it is proposed that the vaporizer device comprises at least one outflow opening, arranged in the vaporizer space housing and/or in the vaporizer surface of the heating unit, for conveying away a non-vaporized portion of the liquid fed to the vaporizer space housing and/or of the aerosol fed to the vaporizer space housing. If the outflow opening is arranged in the vaporizer space housing, the outflow opening preferably has an orientation that runs transversally, in particular at least substantially perpendicularly, to the longitudinal axis. If the outflow opening is arranged in the vaporizer surface of the heating unit, in particular in the heating plate, the outflow opening preferably has an orientation that runs at least substantially parallel to the longitudinal axis. However, other orientations and/or arrangements of the outflow opening in the vaporizer space housing and/or in the heating plate, deemed expedient by someone skilled in the art, are also conceivable. The implementation according to the invention allows attaining a high degree of process reliability in a vaporizer process, in particular in a sterilization process. It is advantageously possible to counteract contamination of the heating unit.

It is also proposed that the vaporizer surface that is assigned to the heating elements has a coating which is realized so as to be resistant against the liquid that is to be vaporized and/or against the aerosol that is to be vaporized, in particular resistant against H2O2. The coating preferably forms the vaporizer surface and is arranged on a side of the heating plate facing towards the vaporizer space. The coating preferably covers the heating elements, or the heating elements, in particular a surface of the heating elements facing toward the vaporizer space, at least partially form the vaporizer surface that is provided with the coating. The implementation according to the invention allows attaining a high degree of process reliability in a vaporizer process, in particular in a sterilization process. It is advantageously possible to counteract an adhesion of non-vaporized liquid and/or non-vaporized aerosol, in particular non-vaporized H2O2.

Moreover, it is proposed that the vaporizer space housing comprises several vaporizer outlets for feeding the liquid vaporized in the vaporizer space and/or the aerosol vaporized in the vaporizer space, in particular an H2O2 vapor-gas mixture, to a packaging element directly or via a distributor, in particular via one or several conduit/s of the production machine. The vaporizer outlets may be oriented transversally, in particular at least substantially perpendicularly, or at least substantially parallel to the longitudinal axis of the housing. The vaporizer outlets may be arranged at the vaporizer space housing so as to be distributed regularly or irregularly along the circumferential direction. The implementation according to the invention allows attaining a high degree of process reliability in a vaporizer process, in particular in a sterilization process. Advantageously, central production of a sterilizing gas mixture is achievable, which can be distributed to different packaging elements or positions of the production machine in a particularly flexible manner.

Beyond this the invention proposes a production machine, in particular a food product filling and/or food product packaging machine, with at least one vaporizer device according to the invention. The production machine may be intended for use in the food product industry or in the medical industry. The production machine may have further components, deemed expedient by someone skilled in the art, in addition to the vaporizer device, like for example a product production device, a product filling device, a product packaging device by means of which the products that are produced or are to be filled are packaged automatically, a product transport device, or the like. The implementation according to the invention allows attaining a high degree of process reliability in a vaporizer process, in particular in a sterilization process. Reliable sterilization of packaging elements into which, for example, products that are to be filled or to be packaged are filled and packaged is advantageously achievable.

The invention furthermore relates to a method for vaporizing a liquid and/or an aerosol, in particular of liquid H2O2 and/or aerosolized H2O2, by means of a vaporizer device, in particular by means of a vaporizer device according to the invention. It is proposed that in at least one meth-od step a plurality of heating elements of a heating unit embodied as a matrix heating unit, which are arranged regularly and/or irregularly in a plane, are regulated and/or monitored separately, in particular individually, wherein the heating elements are arranged at a heating plate of the heating unit, wherein in at least one method step a liquid and/or an aerosol are/is vaporized by means of a surface of the heating plate which forms a vaporizer surface of the heating unit. The features already disclosed for the vaporizer device are to be understood in analogy with reference to the method, such that the features already disclosed with reference to the vaporizer device are to be considered as disclosed with reference to the method as well. The implementation according to the invention allows attaining a high degree of process reliability in a vaporizer process, in particular in a sterilization process. Reliable sterilization of packaging elements into which, for example, products that are to be filled or to be packaged are filled or packaged is advantageously achievable.

The vaporizer device according to the invention, the production machine according to the invention and/or the method according to the invention shall herein not be limited to the application and implementation described above. In particular, in order to fulfill a functionality that is described here, the vaporizer device according to the invention, the production machine according to the invention and/or the method according to the invention may have a number of individual elements, components and units as well as method steps that differs from a number given here. Moreover, with regard to the value ranges given in the present disclosure, values situated within the limits mentioned shall also be considered as disclosed and as applicable according to requirements.

### BRIEF DESCRIPTIONS OF THE DRAWINGS

Further advantages will become apparent from the following description of the drawing. In the drawing exemplary embodiments of the invention are illustrated. The drawing, the description and the claims contain a plurality of features in combination. Someone skilled in the art will purposefully also consider the features separately and will find further expedient combinations.

It is shown in:

FIG. 1 a schematic illustration of a vaporizer device according to the invention,

FIG. 2 a sectional view of the vaporizer device according to the invention, in a schematic illustration,

FIG. 3a a schematic view from above onto a heating unit of the vaporizer device ac-cording to the invention, in a state when a vaporizer space housing is de-mounted from a heating housing of a housing of the vaporizer device according to the invention,

FIG. 3b a schematic view from above onto an alternative heating unit of the vaporizer device according to the invention in state when a vaporizer space housing is demounted from a heating housing of an alternative housing of an alternative-ly implemented vaporizer device according to the invention,

FIG. 4 a schematic illustration of a production machine according to the invention with the vaporizer device according to the invention, and

FIG. 5 a schematic flow chart of a method according to the invention.

### DETAILED DESCRIPTION

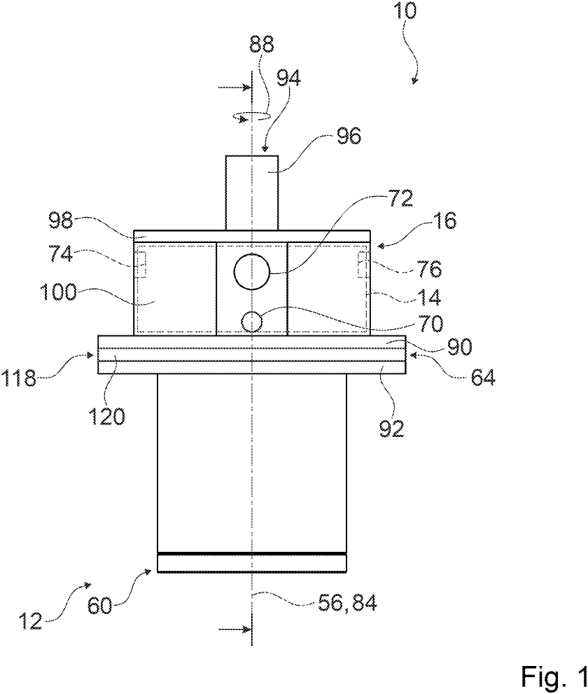

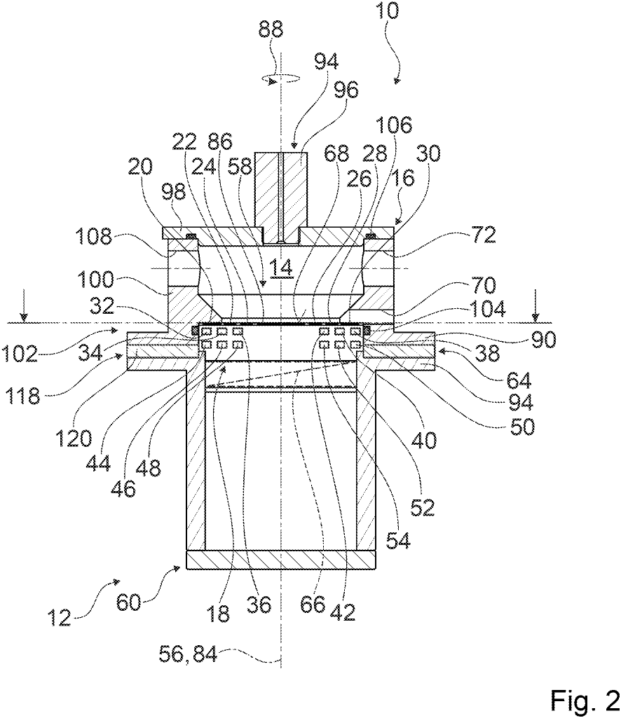

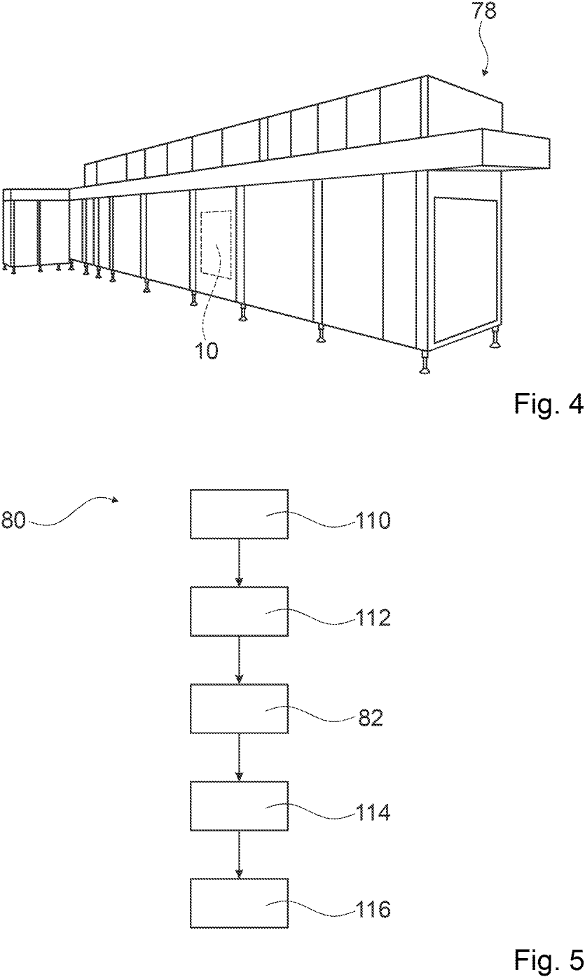

FIG. 1 shows a vaporizer device 10 for a vaporization of a liquid and/or of an aerosol, in particular of liquid H2O2 and/or aerosolized H2O2. The vaporizer device 10 is preferably embodied as a sterilization vaporizer device configured for an application in a sterilization process in the food product industry and/or in the medical industry. The vaporizer device 10 has at least one housing 12, comprising at least one vaporizer space housing 16 which delimits a vaporizer space 14 (see FIG. 2) into which the liquid to be vaporized and/or the aerosol to be vaporized can be fed. The vaporizer device 10 comprises at least one heating unit 18 for a vaporization of the fed-in liquid and/or the fed-in aerosol. The vaporizer device 10 is preferably part of a pro-duction machine 78, in particular a food product filling and/or food product packaging machine, which is exemplarily shown in FIG. 4. It is however also conceivable that the vaporizer device 10 is realized as a self-contained device which can be operated independently from the production machine 78.

The housing 12 of the vaporizer device 10 is preferably realized in a multi-part implementation. The housing 12 is in particular configured for accommodating, at least partly enclosing and/or supporting individual components of the vaporizer device 10. The housing 12 comprises the vaporizer space housing 16 and at least one heating housing 60 which accommodates the heating unit 18 at least partially. Preferentially the vaporizer space housing 16 and the heating housing 60 together delimit the vaporizer space 14. The vaporizer space housing 16 is preferably realized in a bell like shape. The vaporizer space housing 16 may have a round cross section, an elliptic cross section, a polygonal cross section or a different cross section deemed expedient by someone skilled in the art, in particular viewed in a plane that extends at least substantially perpendicularly to a longitudinal axis 84 of the housing 12. The longitudinal axis 84 of the housing 12 preferably runs at least substantially perpendicularly to a vaporizer surface 68 of a heating plate 86 of the heating unit 18 (see FIG. 2). The vaporizer surface 68 is preferably arranged on a side 58 of the heating plate 86 that faces toward the vaporizer space 14. The vaporizer surface 68 of the heating unit 18, which is assigned to heating elements 20, 22, 24, 26, 28, 30 of the heating unit 18, has a coating that is implemented so as to be resistant against the liquid that is to be vaporized and/or the aerosol that is to be vaporized, in particular resistant against H2O2. The vaporizer space housing 16 delimits the vaporizer space 14 preferably at least along a circumferential direction 88 and in at least one direction that runs at least substantially parallel to the longitudinal axis 84, in particular on a side of the vaporizer space 14 that faces away from the heating plate 86. The circumferential direction 88 preferentially runs in the plane extending at least substantially perpendicularly to the longitudinal axis 84 of the housing 12. The vaporizer space housing 16 is preferably made of a metallic material. However, it is also conceivable that the vaporizer space housing 16 is made of a different material, deemed expedient by someone skilled in the art, which is suitable to be used in a vaporizer process, in particular in an H2O2 vaporizer process. Preferentially, the heating housing 60 and the vaporizer space housing 16 are — in particular releasably — connected with each other. Preferably the heating housing 60 and the vaporizer space housing 16 each comprise a connection flange 90, 92 for a connection of the heating housing 60 and the vaporizer space housing 16. For example, the heating housing 60 and the vaporizer space housing 16 are — in particular releasably — connected with each other by means of a screw connection (not shown here in detail) via a connection interface 64 defined by the connection flanges 90, 92. In a state when the heating housing 60 and the vaporizer space housing 16 are connected, the heating plate 86 is arranged between the heating housing 60 and the vaporizer space housing 16, in particular in a region of the connection interface 64. Preferably the vaporizer space housing 16 is implemented so as to be removable from the heating housing 60, in particular for maintenance purposes of the heating unit 18. The connection interface 64 preferably extends in a plane extending transversally, in particular at least substantially perpendicularly, to the longitudinal axis 84.

The vaporizer device 10 preferentially comprises at least one insulation unit 118, which has at least one insulation element 120 that is arranged between the vaporizer space housing 16 and the heating housing 60, in particular for an insulation of electronic components of the heating unit 18 which are arranged in the heating housing 60 against a high heat load (see FIGS. 1 and 2). The insulation element 120 may have a round cross section, a polygonal cross section or a different cross section that is deemed expedient by someone skilled in the art. Viewed along a direction running at least substantially parallel to the longitudinal axis 84, the insulation element 120 is preferentially arranged between the vaporizer space housing 16 and the heating housing 60, in particular in the region of the connection interface 64. Preferentially the insulation element 120 is implemented in a ring shape, in particular in a circular-arc shape.

The vaporizer device 10 preferably comprises at least one feeding unit 94, comprising at least one feeding element 96, in particular a feeding nozzle or a feeding dripper, for a feeding of a liquid and/or an aerosol, in particular a liquid H2O2 and/or an aerosolized H2O2, into the vaporizer space 14. The feeding element 96 is preferably arranged at the vaporizer space housing 16, in particular at a cover 98 of the vaporizer space housing 16 which is situated opposite the heating plate 86. It is however also conceivable that the feeding element 96 is arranged in a different position of the housing 12, deemed expedient by someone skilled in the art, so as to enable a feeding of a liquid and/or an aerosol, in particular a liquid H2O2 and/or an aerosolized H2O2. The feeding element 96 is preferentially arranged at the vaporizer space housing 16 in such a way that a central axis of the feeding element 96 is oriented at least substantially parallel to, in particular coaxially with, the longitudinal axis 84 of the housing 12. The cover 98 of the vaporizer space housing 16 is preferably arranged, in particular fixed, in a removable fashion at a base body 100 of the vaporizer space housing 16, which is in particular cylindrical, preferably circle-cylindrical. However, it is also conceivable that the cover 98 is realized in a one-part implementation with the base body 100 of the vaporizer space housing 16, is in particular free of a releasable connection technique, and/or is arranged at the base body 100 in a loss-proof manner.

The vaporizer space housing 16 preferentially comprises at least one vaporizer outlet 72, 74, 76 or a vaporizer outflow connection for feeding the liquid vaporized in the vaporizer space 14 and/or the aerosol vaporized in the vaporizer space 14, in particular an H2O2 vapor-gas mixture, to a packaging element directly or via a distributor, in particular via one or several conduit/s (not shown here in detail) of the production machine 78. The vaporizer outlet 72, 74, 76 or the vaporizer outflow connection may be oriented transversally, in particular at least substantially perpendicularly, or at least substantially parallel to the longitudinal axis 84 of the housing 12. In at least one implementation of the vaporizer device 10, the vaporizer space housing 16 comprises several vaporizer outlets 72, 74, 76 for feeding the liquid vaporized in the vaporizer space 14 and/or the aerosol vaporized in the vaporizer space 14, in particular an H2O2 vapor-gas mixture, to several packaging elements directly or via a distributor.

FIG. 2 shows a sectional view of the vaporizer device 10 along a plane comprising the longitudinal axis 84. The vaporizer device 10 preferably comprises at least one sealing unit 102, comprising at least one sealing element 104 which is arranged between the vaporizer space housing 16 and the heating plate 86. Viewed along a direction running at least substantially perpendicularly to the longitudinal axis 84, the sealing element 104 is arranged between the vaporizer space housing 16 and the heating plate 86. However, alternatively or additionally it is conceivable that, viewed along a direction that runs at least substantially parallel to the longitudinal axis 84, a sealing element, in particular the aforementioned sealing element, of the sealing unit 102 is arranged between the vaporizer space housing 16 and the heating plate 86. The sealing element 104 preferably adjoins with at least one side the vaporizer space housing 16 and with at least one further side the sealing element 104 adjoins the heating plate 86. The vaporizer space housing 16 and/or the heating plate 86 may comprise at least one receiving recess for receiving the sealing element 104 at least partially. It is however also conceivable that the sealing unit 102 comprises a plurality of sealing elements which are arranged between the vaporizer space housing 16 and the heating plate 86. In an implementation of the vaporizer space housing 16 with a removable cover 98, the sealing unit 102 preferably comprises at least one further sealing element 106 which is arranged between the cover 98 and the base body 100, in the manner shown exemplarily in FIG. 2.

The vaporizer device 10 may comprise an insulation unit having at least one insulation element which is arranged between the vaporizer space housing 16 and the heating housing 60, in particular for an insulation of electronic components of the heating unit 18 which are arranged in the heating housing 60 against a high heat load. The insulation unit may additionally comprise at least one further insulation element, which is arranged on an outer side of the vaporizer space housing 16. The further insulation element may encompass the vaporizer space housing 16 along the circumferential direction 88, in particular so as to counteract a formation of condensate on an inner wall of the vaporizer space housing 16.

The vaporizer space housing 16 preferably comprises a cleaning access opening and/or connection 108 so as to enable a cleaning process of the vaporizer space 14, in particular a CIP process (Cleaning-In-Place process). The cleaning access opening and/or connection 108 may be orient-ed transversally, in particular at least substantially perpendicularly, or at least substantially parallel to the longitudinal axis 84 of the housing 12. The cleaning access opening and/or connection 108 may, alternatively or additionally, form an overflow opening of the vaporizer space housing 16 for conveying away a non-vaporized portion of the liquid fed to the vaporizer space 14 and/or of the aerosol fed to the vaporizer space 14.

The heating plate 86 is preferably arranged at the housing 12 symmetrically, in particular rotationally symmetrically, to the longitudinal axis 84. The heating plate 86 in particular has a main extent that runs at least substantially perpendicularly to the longitudinal axis 84. The vaporizer surface 68 preferably extends at least substantially perpendicularly to the longitudinal axis 84. In particular, in the exemplary embodiment that is shown here, the vaporizer surface 68 is realized as a planar surface. However, it is also conceivable that the vaporizer surface 68 has a different implementation with regard to its contour, deemed expedient by someone skilled in the art, like for example step-like, domed, undulated, or the like.

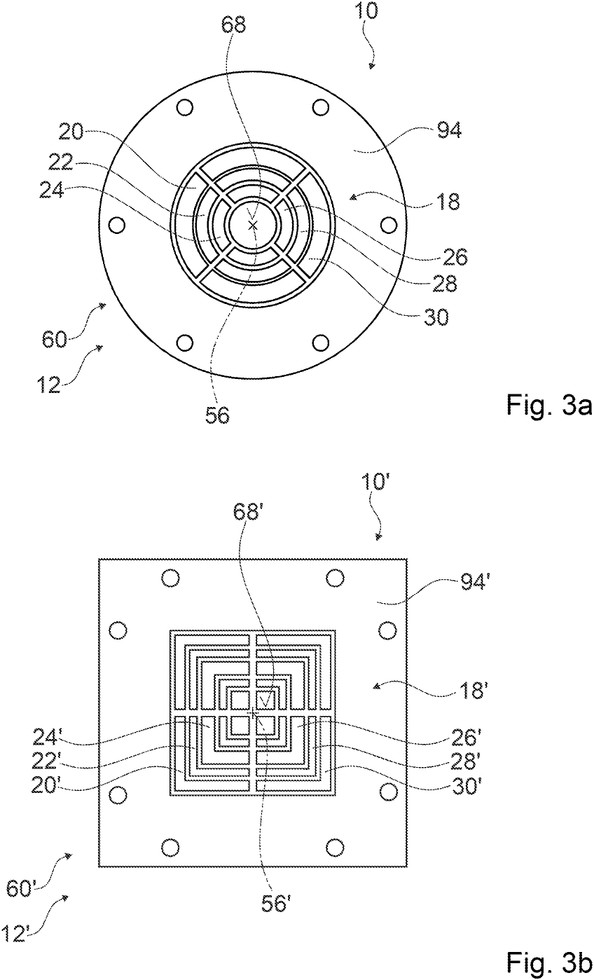

FIG. 3a shows a schematic top view onto the heating unit 18 in a state when the vaporizer space housing 16 is demounted from the heating housing 60. The heating unit 18 is embodied as a matrix heating unit comprising heating elements 20, 22, 24, 26, 28, 30 which can be regulated and/or monitored separately, in particular individually. The heating elements 20, 22, 24, 26, 28, 30 are preferably implemented as heating resistors which can be regulated and/or monitored separately, in particular individually. Preferentially the heating unit 18 comprises a plurality of segmented electrical heating circuits, which are arranged concentrically or in a grid, in particular when viewed in a plane. Preferably a heating element 20, 22, 24, 26, 28, 30, in particular each individual heating element 20, 22, 24, 26, 28, 30, forms an individual heating circuit, in particular in the context of wiring, of the heating unit 18. The heating elements 20, 22, 24, 26, 28, 30 are preferentially arranged at, in particular in, the heating plate 86 of the heating unit 18. The heating elements 20, 22, 24, 26, 28, 30 may be arranged at the heating plate 86 so as to be dis-tributed regularly or irregularly. Preferably the heating elements 20, 22, 24, 26, 28, 30 are inserted in the heating plate 86, in particular embedded in the heating plate 86. Preferably the heating elements 20, 22, 24, 26, 28, 30 terminate at least substantially flush with a surface of the heating plate 86. The surface of the heating plate 86, in particular together with a surface of the heating elements 20, 22, 24, 26, 28, 30, forms the vaporizer surface 68 of the heating unit 18 for a vaporization of a liquid and/or an aerosol, in particular of liquid H2O2 and/or aerosolized H2O2. It is however also conceivable that the heating elements 20, 22, 24, 26, 28, 30 are arranged at, in particular in, the heating plate 86 in a plane that is offset in parallel to a surface of the heating plate 86 which forms the vaporizer surface 68, the heating elements 20, 22, 24, 26, 28, 30 for example contacting the vaporizer surface 68 in order to heat the latter. Other arrangements of the heating elements 20, 22, 24, 26, 28, 30, deemed expedient by someone skilled in the art, at, in particular in, the heating plate 86 are also conceivable.

Each heating element 20, 22, 24, 26, 28, 30 is assigned an, in particular individual, regulation electronics element 32, 34, 36, 38, 40, 42 of the heating unit 18 and/or an, in particular individual, monitoring electronics element 44, 46, 48, 50, 52, 54 of the heating unit 18. Preferably each electrical heating circuit comprises an integrated regulation electronics element 32, 34, 36, 38, 40, 42 of the heating unit 18 and/or an integrated monitoring electronics element 44, 46, 48, 50, 52, 54 of the heating unit 18. The heating elements 20, 22, 24, 26, 28, 30 preferably form, together with the assigned regulation electronics elements 32, 34, 36, 38, 40, 42 and/or the monitoring electronics elements 44, 46, 48, 50, 52, 54 of the heating unit 18, electrical heating circuits of the heating unit 18 which can be regulated and/or monitored individually.

In the exemplary embodiment illustrated in FIG. 3a, the heating elements 20, 22, 24, 26, 28, 30 are arranged concentrically around a central axis 56 of the heating unit 18. In an exemplary embodiment of a vaporizer device 10′ illustrated in FIG. 3b, heating elements 20′, 22′, 24′, 26′, 28′, 30 of a heating unit 18′ of the vaporizer device 10′ are arranged in a grid-like fashion on a side of the heating unit 18′ which faces toward a vaporizer space housing (not shown here in detail). Differently than in the exemplary embodiment of the vaporizer device 10 already described in FIGS. 1 to 3a, the vaporizer device 10′ illustrated in FIG. 3b has an alternative arrangement and/or implementation of the heating unit 18′ as well as an alternative implementation of a housing 12′. The housing 12′ of the vaporizer device 10′ illustrated in FIG. 3b and the heating unit 18′ illustrated in FIG. 3b have a polygonal cross section, in particular a tetragonal or rectangular cross section. However, it is also conceivable that only the heating unit 18′ has a polygonal, in particular tetragonal or rectangular, cross section, the housing 12′ having a round cross section. The other way round is also conceivable. With regard to further features of the alternative implementation of the vaporizer device 10′ illustrated in FIG. 3b, the description given above as well as the following description of the vaporizer device 10 illustrated in FIGS. 1 to 3a may be referred to, such that the features of the vaporizer device 10 are to be read analogously onto the vaporizer device 10′ illustrated in FIG. 3b.

FIG. 2 shows that the housing 12 comprises at least the heating housing 60 for accommodating the heating unit 18, the heating housing 60 being — in particular directly — connected with the vaporizer space housing 16 and the heating elements 20, 22, 24, 26, 28, 30 being arranged at least partially in the region of the connection interface 64 of the housing 12 between the heating housing 60 and the vaporizer space housing 16. The heating unit 18 is arranged at the vaporizer space housing 16 in such a way that at least one heating element 20, 30 of the heating unit 60 — in particular directly — adjoins the vaporizer space housing 16. Preferably at least two, in particular at least four, heating elements 20, 30 of the heating unit adjoin the vaporizer space housing 16 directly. Preferentially the heating elements 20, 30 arranged in a peripheral region of the heating plate 86 adjoin the vaporizer space housing 16 directly.

The vaporizer device 10 further comprises at least one elastic preload element 66 (shown in FIG. 2 by a dashed line), in particular a spring element, which is configured to subject the heating unit 18 to a preload force toward the vaporizer space housing 16. The preload element 66 is preferentially supported with one side on the heating unit 18, and with a further side the preload element 66 is supported on the heating housing 60, in particular on an inner wall of the heating housing 60. The preload element 66 may be embodied as a helical spring, as an elastomer, as a gas compression spring, as a leaf spring or as another preload element that is deemed expedient by someone skilled in the art.

The vaporizer device 10 comprises at least one outflow opening 70 (see FIGS. 1 and 2), which is arranged in the vaporizer space housing 16 or in the vaporizer surface 68 of the heating unit 18 for conveying away a non-vaporized portion of the liquid fed to the vaporizer space housing 16 and/or the aerosol fed to the vaporizer space housing 16. The outflow opening 70 preferentially has an orientation at least substantially parallel to the longitudinal axis 84. However, different orientations and/or arrangements, deemed expedient by someone skilled in the art, of the outflow opening 70 in the vaporizer space housing 16 and/or in the heating plate 86 are also conceivable. It is further conceivable that, in addition to conveying away a non-vaporized portion of the liquid fed to the vaporizer space housing 16 and/or the aerosol fed to the vaporizer space housing 16, the outflow opening 70 is configured for a connection of a cleaning device, in particular a CIP cleaning device.

FIG. 5 shows a flow chart of a method 80 for vaporizing a liquid and/or an aerosol, in particular liquid H2O2 and/or aerosolized H2O2, by means of the vaporizer device 10. In at least one method step 110, the heating unit 18 is activated. In at least one method step 112, a liquid and/or an aerosol are/is fed to the vaporizer space 14 via the feeding unit 94. In at least one method step 82, the heating elements 20, 22, 24, 26, 28, 30 of the heating unit 18 that is realized as a matrix heating unit are regulated and/or monitored separately, in particular individually. The heating elements 20, 22, 24, 26, 28, 30 are regulated and/or monitored separately by means of the respective regulation electronics element 32, 34, 36, 38, 40, 42 and/or monitoring electronics element 44, 46, 48, 50, 52, 54. In at least one method step 116, suction of the liquid vaporized in the vaporizer space 14 and/or the aerosol vaporized in the vaporizer space 14, in particular the H2O2 vapor-gas mixture, is carried out, in particular via conduits, for a feeding to packaging elements which are to be sterilized. In at least one method step 114, which is in particular not necessarily present, a substance concentration, in particular an H2O2 concentration, in the liquid vaporized in the vaporizer space 14 and/or in the aerosol vaporized in the vaporizer space 14 is registered, in particular for a regulation of the heating unit 18 that is dependent thereon, and/or drop detection and/or, in particular indirect, determination of a liquid vaporized in the vaporizer space 14 and/or of the aerosol vaporized in the vaporizer space 14, in particular the H2O2 vapor-gas mixture, are/is carried out via a power consumption of the heating unit 18. Regarding further method steps of the method 80, the description of the vaporizer device 10 is referred to, which is to be understood analogously for the method 80.

1. A vaporizer device for vaporizing a liquid and/or an aerosol, with at least one housing (12) comprising at least one vaporizer space housing (16), which delimits a vaporizer space (14) into which the liquid to be vaporized and/or the aerosol to be vaporized can be fed, and with at least one heating unit (18) for a vaporization of the fed-in liquid and/or the fed-in aerosol, wherein the heating unit (18) is realized as a matrix heating unit comprising a plurality of heating elements (20, 22, 24, 26, 28, 30), which can be regulated and/or monitored separately, wherein the heating unit (18) comprises the plurality of heating elements (20, 22, 24, 26, 28, 30), which are arranged irregularly and/or regularly in a plane, wherein the plurality of heating elements (20, 22, 24, 26, 28, 30) are arranged at a heating plate (86) of the heating unit (18), wherein a surface of the heating plate (86) forms a vaporizer surface (68) of the heating unit (18) for a vaporization of the liquid and/or of an aerosol.

2. The vaporizer device according to claim 1, wherein each heating element (20, 22, 24, 26, 28, 30) is assigned a regulation electronics element (32, 34, 36, 38, 40, 42) of the heating unit (18) and/or monitoring electronics element (44, 46, 48, 50, 52, 54) of the heating unit (18).

3. The vaporizer device according to claim 1 wherein the heating elements (20, 22, 24, 26, 28, 30) are arranged concentrically around a central axis (56) of the heating unit (18), or that the heating elements (20, 22, 24, 26, 28, 30) are arranged in a grid-like fashion on a side (58) of the heating unit (18) that faces toward the vaporizer space housing (16).

4. The vaporizer device according to claim 1, wherein the housing (12) comprises at least one heating housing (60) for an accommodation of the heating unit (12), wherein the heating housing (60) is connected with the vaporizer space housing (16) and the heating elements (20, 22, 24, 26, 28, 30) are arranged at least partly in the region of a connection interface (64) of the housing (12) between the heating housing (60) and the vaporizer space housing (16).

5. The vaporizer device according to claim 1, wherein the heating unit (18) is arranged at the vaporizer space housing (16) in such a way that at least one heating element (20, 30) of the heating unit (18) is adjacent to the vaporizer space housing (16).

6. The vaporizer device according to claim 1, further comprising at least one elastic preload element (66) which is configured to subject the heating unit (18) to a preload force toward the vaporizer space housing (16).

7. The vaporizer device according to claim 1, further comprising at least one outflow opening (70), arranged in the vaporizer space housing (16) and/or in the vaporizer surface (68) of the heating unit (18), for conveying away a non-vaporized portion of the liquid fed to the vaporizer space housing (16) and/or of the aerosol fed to the vaporizer space housing (16).

8. The vaporizer device according to claim 1, wherein the vaporizer surface (68) of the heating unit (18) that is assigned to the heating elements (20, 22, 24, 26, 28, 30) has a coating which is realized so as to be resistant against the liquid that is to be vaporized and/or the aerosol that is to be vaporized.

9. The vaporizer device according to claim 1, wherein the vaporizer space housing (16) comprises several vaporizer outlets (72, 74, 76) for feeding the liquid vaporized in the vaporizer space (14) and/or the aerosol vaporized in the vaporizer space (14) to a packaging element directly or via a distributor.

10. A production machine with at least one vaporizer device according to claim 1.

11. A method for vaporizing a liquid and/or an aerosol by a vaporizer device according to claim 1 wherein in at least one method step (82) the plurality of heating elements (20, 22, 24, 26, 28, 30) of the heating unit (18) embodied as the matrix heating unit, which are arranged irregularly and/or regularly in a plane, are regulated and/or monitored separately, wherein the plurality of heating elements (20, 22, 24, 26, 28, 30) are arranged at the heating plate (86) of the heating unit (18), wherein in at least one method step a liquid and/or an aerosol are/is vaporized by means of the surface of the heating plate (86) which forms the vaporizer surface (68) of the heating unit (18).

12. The vaporizer device according to claim 6, wherein the at least one elastic preload element (66) is a spring element.
